(19) United States
(12) Patent Application Publication
(10) Pub. No.: US 2005/0196461 A1
(43) Pub. Date: Sep. 8, 2005

(54) Ceramide formulations suitable for oral administration

(76) Inventor: Ronald G. Udell, Beverly Hills, CA (US)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/035,120

(22) Filed: Jan. 13, 2005

Related U.S. Application Data
(60) Provisional application No. 60/536,334, filed on Jan. 14, 2004.

(51) Int. Cl. A61K 31/739; A61K 31/728; A61K 31/4188; A61K 35/32; A61K 35/78

(52) U.S. Cl. 424/549; 514/54; 514/393; 424/750

(57) ABSTRACT

The present invention is directed to compositions and methods of delivery of ceramides and additives in a formulation, such as a soft gelatin capsule, suitable for oral delivery.

### CROSS REFERENCE TO RELATED APPLICATION

This application claims benefit under 35 U.S.C. § 119(e) to application Ser. No. 60/536,334, filed Jan. 14, 2004, entitled “Soft Gelatin Capsules Containing Ceramides” (attorney docket number 33917/US), the contents of which are incorporated herein in their entirety for all purposes.

### FIELD OF THE INVENTION

The present invention relates to orally administered formulations containing ceramides for skin protection, for example, for treatment of wrinkles, fine lines and/or skin dryness.

### BACKGROUND OF THE INVENTION

Ceramides are sphingolipids present in all cell structures. Ceramides are constituents of cells in the cytoplasmic membranes. Thus, they are found in the skin, central nervous system and spinal marrow. Ceramides are also present in the plant world, with the main sources being wheat, rice, soy, and spinach. These compounds are the result of the formation of an amide bond between a fatty acid and sphingosine, or phytosphingosine. The amounts of ceramides in food vary considerably, and the per capita ceramide consumption in the United States is estimated to be 0.3 to 0.4 grams per day.

Ceramides are found naturally in many foods and are commercially derived from plant sources. Two popular sources are brown rice and wheat germ extracts. These are the two common forms of commercially available ceramide for dietary supplement and cosmetic applications.

Ceramides are commercially extracted from rice bran or wheat germ. These plant sources contain a large amount of glycosphingolipids. The glycosphingolipids of rice bran is similar to animal glycosphingolipids, in which the backbone of the ceramide including sphingoid bases with fatty acids is an amide linkage, and the terminal hydroxyl group is substituted by glucose. Glycosphingolipids have a great variety of molecular species because of the partial structure of sphingoid bases.

Ceramides are natural components of human skin. There are six ceramide species commonly found in skin. These ceramides are formed via a biosynthetic mechanism in the epidermis. The four layers of the epidermis contain ceramides and they play an important role by creating a barrier that reduces infection and helps retain the skin's moisture. Reduction in the amount of ceramides in the epidermal layers results in dry skin, dermatitis, and is believed to be a major cause of wrinkles. Studies have shown that a proper amount of ceramides in the internal epidermal layer is necessary to maintain healthy skin.

Currently available ointments, creams, pastes, and lotions that contain ceramides can be applied topically to the skin to help reduce the afore-mentioned afflictions and to maintain healthy skin. However, a product that delivers significant quantities of ceramides orally is not readily available.

There is a need in the art for an orally administrable ceramide composition that is capable of delivering an effective amount of a ceramide that prevents, reduces or minimizes the afore-mentioned skin afflictions.

### BRIEF SUMMARY OF THE INVENTION

The present invention pertains to ingestible compositions that includes a ceramide or an analog thereof as an active ingredient and an acceptable carrier. In one aspect of the invention, the carrier is not a silicone polymer. In another aspect of the invention, the ceramide is a phyto-derived ceramide.

The ceramide compositions of the invention can further include additives such as hyaluronic acid and/or biotin.

Suitable carriers for the ingredients include, for example, pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); and/or disintegrants (e.g., potato starch or sodium starch glycolate).

The present invention also provides packaged nutraceutical formulations that include a composition comprising a ceramide and a carrier; and instructions for use thereof.

The present invention also pertains to an ingestible soft gelatin composition that includes a ceramide or an analog thereof as an active ingredient; and an acceptable carrier. In one aspect of the invention, the carrier is not a silicone polymer. In another aspect of the invention, the ceramide is a phyto-derived ceramide.

The encapsulated soft gelatin ceramides of the invention can further include additives such as hyaluronic acid and/or biotin.

Suitable carriers for the ingredients include, for example, wheat germ oil or yellow beeswax.

The present invention also provides methods to treat dry skin, wrinkles, melanogenesis, atopic dermatosis, psoriasis and/or fine lines with the compositions of the invention.

The present invention also provides packaged nutraceutical formulations that include a soft gelatin capsule comprising a ceramide and a carrier; and instructions for use thereof.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive.

### DETAILED DESCRIPTION

The present invention pertains to the surprising discovery that ceramides can be formulated into ingestible compositions, such as soft gelatin capsules, in an acceptable carrier. The ceramide compositions of the present invention can be taken orally, and are useful for maintenance of healthy skin. For example, the formulations of the invention can be used to treat, prevent and/or reduce afflictions mentioned throughout the specification, such as dry skin, wrinkles and fine lines in the skin. Until the time of this invention, ceramide(s) was/were generally provided in topical formulations. However, it has been found that oral administration of a ceramide can be advantageous for skin health.

The term “ceramide” is recognized in the art and is related to sphingolipids that are present in cell structures. Ceramides are also notably present in the plant world and in particular in wheat, rice, soya, millet, olive and spinach. Alternatively, ceramides can also be synthesized.

The term ceramide used in the present specification is recognized by a person skilled in the art. The term ceramide, therefore, includes lipids composed of the sphingosine family, such as sphinganine, 4-hydroxy-sphinganine or phytosphingosine, which are bonded to a fatty acid or fatty acid derivative via their amine functional group. The term ceramide thus includes all ceramides, of synthetic or natural origin (vegetable, animal or human) optionally substituted, for example, by a sugar such as mono- or polyglucosylceramides.

The term “processed ceramide” refers to a ceramide or ceramide analog that is isolated from its non-naturally occurring state, such as the naturally occurring ceramide found in wheat or rice. That is to say, processed ceramides include the ceramide oils, powders, gums, etc. isolated from treated natural sources that contain ceramide, such as wheat, oryza, rice etc. (pressed, extracted, distilled, fractionated, etc.).

The ceramides of the stratum corneum are composed of 6 chromatographically distinct fractions having a different polarity according to the degree of unsaturation (which may be zero) or of hydroxylation of their chains, their length and their number. The ceramides are categorized according to their chemical configuration as class I, II, III, IV, V, VIa and VIb. Their chemical configuration is in particular provided in the document “Ceramides, Key Components for Skin Protection,” by R. D. Petersen, Cosmetics & Toiletries, vol. 107, February 1992, p. 45-49 and the document in EJD, No. 1, vol. 1, October 1991, Review article, p. 39-43, “Skin Ceramides: Structure and Function,” by M. Kerscher, the contents of which are incorporated herein in their entirety.

As stated above, ceramides used in the present invention are amide derivatives in which fatty acids are attached to the amine groups of sphingosine, phtyosphingosine, or sphinganine, exemplified by the following structures: 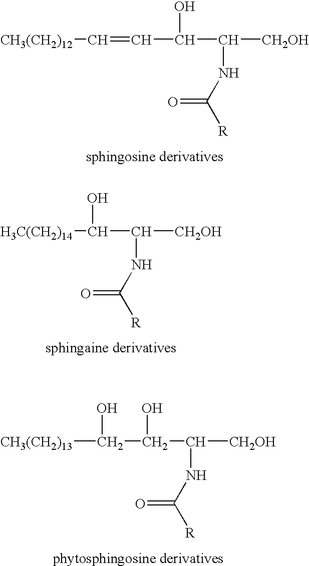
wherein R is a C₆-C₂₅ unsaturated or saturated carbon chain which has one or more double bonds or a C₆-C₂₅ unsaturated or saturated carbon chain with a hydroxy group at the alpha or beta position.

Plants contain structures, which are chemically similar to human ceramides. These ceramide products can help in creating the protective barrier in the epidermis. Supplementation with an oral ceramide composition replaces the components lost with aging. The moisturizing effect directly comes from the ceramides being carried directly to the stratum corneum via the blood. This direct method improves the functionality of the agent and produces results not seen in cosmetic topical applications.

The term ceramide also includes oryza ceramide (derived from oryza sativa), a brown extract from rice bran or rice germ that contains large amounts of glycosphingolipid. Both glycosphingolipids of rice bran and those derived from animal sources include an sphingoid base with a fatty acid amide linkage, with the terminal hydroxyl group being substituted by glucose (or another sugar moiety). Oryza ceramides are commercially available from companies such as Oryza Oil & Fat Co. Ltd., Japan. Likewise, phyto-ceramides are extracted from Konjac-tuber (Amorphophallus konjac) and are glycosylated ceramides. Konjac-tuber ceramide is commercially available from Unitika Ltd, Kyoto, Japan. Another suitable ceramide source is “OptiPure®” Ceramide from Chemco Industries, Los Angeles, USA.

For example, in one embodiment, ceramide is derived from a special fraction of plant based product. The ceramide itself is extracted with ethanol at ninety-nine degrees Celsius. The flour fraction is incorporated into the ethanol under a rapid mechanical stirring for two hours. The whole separation is transferred into a Buchner filter. The process then moves into a clarification of the filtrate, which consists of wheat lipids and ethanol. After large insoluble particles are removed, the extraction solvent is distilled into a reactor distillatory. Separation of polar liquids and apolar liquids take place. The oil is then mixed in hot water with an emulsor during 30 minutes and the mixture is allowed to settle overnight. The polar lipids and particularly, the sphingolipids and glycosphingolipids form a stable emulsion in water. During the recovery phase, apolar liquids have a density inferior to water and rise to the surface of the mixture; so water containing polar lipids can be collected by gravity filtration. The water fraction is then freeze dried, resulting in a powder which is crushed and micronized, packed into hermetically sealed bags with a desiccant.

Therefore, it should be understood that the terms “ceramide” or “ceramide analog thereof” are inclusive of all types of ceramides described throughout the present specification, including sugar substituted (glycosylated) and those as are known in the art.

The stratum corneum is the outermost layer of mammalian skin. This layer primarily acts as a barrier to protect us from external environmental stresses and to prevent excessive transcutaneous water loss. The cells of the stratum corneum, comeocytes, and the lipids between them, ceramides, accomplish this important function. Disruptions of this barrier through either physical trauma caused by dermatitic conditions like eczema results in this important function being compromised. The result is decreased elasticity, increased susceptibility to infection, and increased water loss which can lead to aging conditions like such as wrinkles and fine lines as well as an overall dullness to the skin's texture.

Ceramides have become an important compound for skin protection. Lipid depletion and replenishment studies have shown that ceramides play an essential role in establishment and maintenance of the water-retaining properties of skin. Since it is known that the natural levels of ceramides produced by an individual decrease with age, it has been suggested that increased transepidermal water loss is the result of their reduced presence in the skin. Ceramides play an important role in preventing moisture loss that can be caused by physical trauma or aging.

It is also know that ceramide inhibits melanogenis and is thought to promote a pigment lightening effect. Melanogenis is a process by which the skin produces pigments that give our skin tone and color. This is particularly helpful for hyper-pigmentation that causes age spots and other discolorations of the skin.

Studies show that ceramide can be absorbed via the digestive system and carried to the stratum corneum by blood, where it circulates, and produces a significant effect in promoting a water barrier in the layers of the skin. Therefore, ceramides in the stratum corneum of the skin play important roles for maintaining a barrier function and protecting the skin from harm.

Much has been said about “anti-aging” creams and lotions and more has been sold based on the current marketing of beauty creams, which promise youthful skin. Aggressive marketing and promising cosmetic formulas have characterized much of this sales boom. The term anti-aging is important in establishing the market for ceramide products. In the early 1990's, major cosmetic companies such as Elizabeth Arden formulated and branded products based on the safe and effective use of ceramides for treating aging problems such as fine lines, wrinkles, and dryness. These were often in the form of expensive topical creams and potions. Because of moisture retaining properties, ceramides were eventually included in cosmetic products such as foundations and lipsticks to increase outer moisture and provide a benefit to the skin while protecting it from damaging forces.

Currently there are many types of ceramides on the market. Originally ceramides were derived from soybeans and bovine sources. Now, fermentation processes from yeast and glycerol are now available. Topical application of processed ceramides can result in depth reduction of wrinkles in some people, as well as smoother skin due to an increase in the water content of the skin. This is not unusual since the layers of skin already contain ceramide. Dry and aging skin shows the most improvement with continued topical ceramide use. In addition, people with psoriasis or Atopic Dermatitis may have low levels of ceramide in their skin. This is thought to produce the damage caused by these skin impairing conditions.

Ceramide use is so promising that in the August 2001 issue of “The Archives of Dermatology” (Vol. 137, number 8) a report that children with Atopic Dermatitis showed improvement with topically applied products containing different ceramides as well as other fatty acids. This has further enhanced the appeal for ceramide as a necessary nutrient for skin health. The present formulations provided throughout the specification can be used to treat this condition.

Internal ingestion of ceramide is a concept, which takes the beneficial effects of ceramides to a new level. While a topical cream can provide only so much of a moisture layer, an internal formula can really be translated to the cell level, thereby increasing the potential for the product to be absorbed into the layers of the skin.

There are no special requirements for the use in dietary supplements containing ceramides, such as the need to take it on an empty stomach. Ceramides can be taken for a long term regimen to keep the skin moist and healthy. The recommended dose is between about 20 mg and about 600 mg per day, more particularly between about 30 mg and about 300 mg per day, and in particular between about 30 mg to about 50 mg a day for a whitening effect on the skin.

For a moisturizing effect, the recommended dose of ceramide is between about 10 mg and about 600 mg per day, more particularly between about 20 mg and about 400 mg per day, and in particular between about 20 mg and 50 mg per day. An oral formulation containing ceramide provides a cost effective way to get more ceramide into the skin without the barrier of creams and lotions. The present invention is an internal cosmeceutical application that should revolutionize this safe product. Many products such as Vitamin C, CoQ1O, Vitamin A, E & D have taken the opposite route and are being incorporated into products that are topical and external.

The present invention provides the ability to deliver ceramide(s), as defined herein, in concentrations of up to about 60% (weight to total weight of soft gelatin capsule).

Formulation of the ceramide into a soft gel capsule can be accomplished by many methods known in the art. Often the formulation will include an acceptable carrier, such as an oil, or other suspending agent.

Suitable carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The formulations of the invention are considered dietary supplements useful to the increase the amounts of ceramide(s) in the individuals in need thereof.

Alternatively, the formulations of the invention are also considered to be nutraceuticals. The term “nutraceutical” is recognized in the art and is intended to describe specific chemical compounds found in foods that may prevent disease or ameliorate an undesirable condition. Ceramides are such compounds.

The formulations of the invention can further include various ingredients to help stabilize, or help promote the bioavailability of the ceramide(s), or serve as additional nutrients to an individual's diet. Suitable additives can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Various additives can be incorporated into the present compositions. Optional additives of the present composition include, without limitation, hyaluronic acid, phospholipids, L-carnitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Compositions comprising the active components of the invention, i.e. a ceramide, may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries that facilitate processing of the active compounds into preparations that can be used.

The active compound(s) thereof can be formulated in the pharmaceutical compositions per se, or in the form of a hydrate, solvate, or acceptable salt. Typically, such salts are more soluble in aqueous solutions than the corresponding free acids and bases, but salts having lower solubility than the corresponding free acids and bases may also be formed.

The compositions of the invention may take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampoules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the compositions of the invention may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, cremophore™ or fractionated vegetable oils); and preservatives (e.g., methyl or propyl p hydroxybenzoates or sorbic acid). The preparations may also contain buffer salts, preservatives, flavoring, coloring and sweetening agents as appropriate.

Preparations for oral administration may be suitably formulated to give controlled release of the active compound, as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For nasal administration or administration by inhalation or insufflation, the active compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

Alternatively, other delivery systems may be employed. Liposomes and emulsions are well-known examples of delivery vehicles that may be used to deliver active compound(s). Certain organic solvents such as dimethylsulfoxide (DMSO) may also be employed, although usually at the cost of greater toxicity.

The compositions may, if desired, be presented in a pack or dispenser device, which may contain one or more unit dosage forms containing the active compound(s). The pack may, for example, comprise metal or plastic foil, such as a blister pack. The pack or dispenser device may be accompanied by instructions for administration.

Soft gel or soft gelatin capsules can be prepared, for example, without limitation, by dispersing the formulation in an appropriate vehicle (e.g. rice bran oil, monoterpene and/or beeswax) to form a high viscosity mixture. This mixture is then encapsulated with a gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (rice bran oil or wheat germ oil and/or beeswax if desired) and can include, apart form the antioxidant actives, a hydrophilic matrix. The hydrophilic matrix, if present, is a polyethylene glycol having an average molecular weight of from about 200 to 1000. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams.

For example, a soft gel capsule can be prepared by mixing ceramide oil with D-biotin, natural hyaluronic acid (C-ME), yellow beeswax and wheat germ oil, cold pressed, 3.5-4.7 IU. The mixture is then encapsulated within a soft gelatin capsule as described above.

Emulsifying agents that can be used to help solubilize the ingredients within the soft gelatin capsule include a surfactant, emulsifier, or effervescent agent. These include, for example, D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

The present invention also provides packaged formulations of a ceramide in a soft gel capsule and instructions for use of the product for skin treatment(s). Typically, the packaged formulation, in whatever form, is administered to an individual in need thereof that requires an increase in the amount of ceramide in the individual's diet. Typically, the dosage requirement is between about 1 to about 4 dosages a day.

The active compound(s) of the invention, or compositions thereof, will generally be used in an amount effective to achieve the intended result, for example in an amount effective to treat or prevent the particular skin condition being treated. The composition may be administered therapeutically to achieve therapeutic benefit or prophylactically to achieve prophylactic benefit. By therapeutic benefit is meant eradication or amelioration of the underlying condition being treated and/or eradication or amelioration of one or more of the symptoms associated with the underlying disorder such that the patient reports an improvement in feeling or condition, notwithstanding that the patient may still be afflicted with the underlying disorder. For example, administration of a composition of the invention to a patient suffering from wrinkling of the skin provides therapeutic benefit not only when the underlying condition is eradicated or ameliorated, but also when the patient reports a decrease in the severity or duration of the physical, appearance or emotional discomfort associated with the skin condition.

For prophylactic administration, the composition may be administered to an individual at risk of developing one of the previously described conditions. For example, a person can begin taking the ceramide compositions of the invention in the person's twenties to avoid such skin conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art.

Total dosage amounts will typically be in the range of from about 0.0001 or 0.001 or 0.01 mg/kg/day to about 100 mg/kg/day, but may be higher or lower, depending upon, among other factors, the activity of the components, its bioavailability, the mode of administration and various factors discussed above. Dosage amount and interval may be adjusted individually to provide plasma levels of the compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

In one aspect, the composition of the invention includes between about 5 milligrams (mg) and about 100 mg, more particularly between about 10 mg and about 50 mg, and between about 12 mg and about 25 mg of ceramide per dosage, i.e., between about 10 mg and 30 mg. As previously discussed, the dosing can be administered by any number of delivery methods, i.e., soft gel capsules, tablets, in a foodstuff.

In one embodiment, the gelatin used to prepare the soft gelatin capsule includes gelatin from lime or acid derived gel manufacturing processes known in the art. The gelatin is combined with plasticizers, such as glycerin, sorbitol or other polyalcoholic compounds, or combinations thereof and purified water. Optional additives can include colorants, preservatives, flavors, sweetening agents and/or opacifying agents. The amount of gelatin in the mixture can range from about 30 to about 60 percent (by weight), with about 15 to about 55% plasticizer (by weight) and purified water from about 15 to about 40% by weight. Optional additives are generally present in a range from about 0.1 to about 15% by weight.

A soft gel capsule is prepared by mixing the ceramide and optional components, for a period of time until the mixture is thoroughly mixed, optionally under vacuum. A gelatin mixture is fed into two spreader boxes, which in turn form two gelatin ribbons that are used to make each half of the gelatin capsule shell. The fill mixture (ceramide, hyaluronic acid, bees wax and wheat germ oil, as an example) is pumped into the gelatin ribbons held in place by two rotating die cavity rolls. The capsules are half sealed when a pump injects the fill material into the die cavities. The injection is followed by forming hermetic seals between the two capsule halves and the capsules are cut from the gelatin ribbon.

The capsules are dried at a temperature of a range of 70 to about 75° F. at a relative humidity of between about 15 and about 30 percent. Upon equilibration with the surrounding environment, the dried capsules will have a moisture content of between about 5 and about 10% by weight.

Although the present invention describes the preparation, use, manufacture and packaging of ceramides in soft gelatin capsules for treatment of various skin afflictions, it should not be considered limited to only soft gelatin capsules. Ingestible ceramides can be delivered in traditional tablets, pills, lozenges, elixirs, emulsions, hard capsules, liquids, suspensions, etc. It has only recently been discovered that ceramides are orally acceptable and can be ingested without harm to the individual. Chemco Industries (OptiPure Ceramide) provides such a non-toxic ceramide useful for such formulations.

The following examples are intended to be illustrative only and should not be considered limiting.

### EXAMPLES

Formulations of ceramide and additives, such as hyaluronic acid and/or biotin, can be prepared in the following ratios by mixing the components together and then encapsulating into a soft gel capsule.

| Component | Example 1 | |
|---|---|---|
| Ceramide Oil, MIN 15% (Ennagram, France) | 125 | Kg |
| D-Biotin, Vitamin H | 0.5130 | Kg |
| Natural Hyaluronic Acid | 70.0 | Kg |
| Yellow Beeswax | 15.0 | Kg |
| Wheat Germ Oil, Cold | 289.487 | Kg |
| Pressed, 3.5-4.7 IU | | |
| Total weight | 500 | Kg |

Total weight of fill material in each soft gelatin capsule was between about 500 and 510 mg weight of a total capsule weight of about 530 mg.

A double blind, randomized, placebo controlled, parallel design clinical trial which was conducted at one clinical site.

Twenty females who were between 40 and 65 years of age, in good general health, and had mild to very moderate wrinkling of the skin of the periorbital area were enrolled and randomized. Subjects were required to make a total of four visits to the study site over a 16-week treatment period.

Visit 1 was for screening and enrollment. At this visit, medical history and information regarding concomitant medications were collected. Vital signs were measured and a dermatological examination was conducted. Subjects completed Self-Assessment Questionnaires. Qualified subjects were enrolled and randomized to use either the Soft Gel combination or the placebo combination test products for a 16-week period. Subjects were instructed to consume two softgels in the morning and two softgels in the evening for a total of four softgels each day, and apply a topical cream to the periorbital area twice a day, once in the morning and once in the evening. Photographic images of the facial area, concentrating on the periorbital area, of the subjects were obtained. Subjects were instructed to complete Self-Assessment Questionnaires in their diaries on Day 14.

At each of Visits 2, 3, and 4, information on concomitant illnesses, concomitant medications, and the occurrence of any adverse events were documented. A dermatological examination was conducted by the investigator. The investigator also assigned a global improvement score based on the appearance of the skin of the periorbital area. Subjects completed Self-Assessment Questionnaires. At Visit 3, photographic images of the facial area, concentrating on the periorbital area, of the subjects were repeated.

Twenty-three individuals were recruited. Twenty subjects were qualified and enrolled, and eighteen subjects subsequently completed the study.

Visit 1 (Day 1/Screening)

Visit 1 was for screening and enrollment. At this visit, medical history and information regarding concomitant medications was collected. Vital signs (heart rate, blood pressure and temperature) were measured and a dermatological examination, consisting of an evaluation of the periorbital area was conducted. Subjects completed Self-Assessment Questionnaires. Urine β-HCG pregnancy tests were performed on urine specimens from all women of childbearing potential. Qualified subjects were enrolled and randomized to use either the Soft Gel combination or the placebo combination test products for a 16-week period. They were instructed to consume two softgels in the morning and two softgels in the evening, regardless of meals, for a total of four softgels each day, and apply a topical cream to the periorbital area twice a day, once in the morning and once in the evening. The topical cream was only to be applied after the face, including the periorbital area, was cleansed with subject's normal cleanser. If it became necessary to rewash the face following application of the topical treatment, subject was to allow at least one hour before rewashing. At this visit, subjects were given a four-week supply of test products and a subject diary. Photographic images of the facial area, concentrating on the periorbital area, of the subjects enrolled were obtained. Subjects were instructed to complete Self-Assessment Questionnaires in their diaries on Day 14.

Soft Gel Combination (a and b) Ceramide Enriched

a. Oral:

Wheat Germ Oil, Gelatin, Glycerin, Ceramide Oil, Natural Hyaluronic Acid, Purified Water, Precept 8120 (Powder Lecithin), Sorbitan Mono Oleate, Yellow Beeswax, D-Biotin. The formulation comprised:

| | Mg/capsule | |
|---|---|---|
| Sorbitan Monooleate | 25 | |
| Hyaluronic Acid | 70 | |
| Ceramide Oil (15%) (Ennagram, France) | 83.334 | |
| D-Biotin | 0.26 | |
| Purified DI Water | 37.1 | |
| Wheat Germ Oil | 204.306 | |
| Yellow Beeswax | 20 | |
| Precept 8120 | 60 | |
| Total Fill Weight | 500 | mg |
| Gelatin | 191.363 | |
| Glycerin | 92.151 | |
| Purified Water | 28.650 | |
| Cochineal Extract | 2.202 | |
| Titanium Dioxide | 3.965 | |
| Total Capsule Weight | 818 | mg |

b. Topical:

Water, Caprylic/Capric Triglyceride, Butylene Glycol, Cetyl Alcohol, PEG-12 Glyceryl Distearate, Collagen, Cyclomethicone, Stearyl Alcohol, Cyclopentasiloxane, Glyceryl Stearate, PEG-100 Stearate, Aloe Vera Leaf Gel, Cyanocobalamin, Biotin, Squalane, PEG-40 Stearate, Copper Gluconate, Ceramide (provided by Soft Gel), Sodium PCA, Polyacrylamide, C13-14 Isoparaffin, Hyaluronic Acid (provided by Soft Gel), Laureth-7, Dimethicone/Vinyl Dimethicone Crosspolymer, Disodium EDTA, Fragrance, Methylparaben, Phenoxyethanol, Propylparaben. (BioZone Laboratories Inc., 580 Garcia Avenue, Pittsburg, Calif. USA).

Placebo Combination (a and b) Ceramide Devoid

a. Oral:

Wheat Germ Oil, Gelatin, Glycerin, Yellow Beeswax, Purified Water, Titanium Dioxide, Cochineal Extract. The formulation comprised:

| | Mg/capsule | |
|---|---|---|
| Wheat Germ Oil | 467 | |
| Yellow Beeswax | 33 | |
| Total Fill Weight: | 500 | mg |
| Gelatin | 191.363 | |
| Glycerin | 92.151 | |
| Purified Water | 28.650 | |
| Cochineal Extract | 2.202 | |
| Titanium Dioxide | 3.965 | |
| Total weight of capsule | 818 | mg |

b. Topical:

Water, Butylene Glycol, Cetyl Alcohol, Stearyl Alcohol, Sodium Lauryl Sulfate, Cyanocobalamin, Polyacrylamide, C13-14 Isoparaffin, Laureth-7, Yellow 5, Fragrance, lmidazolidinyl Urea, Methylparaben, Propylparaben, Phenoxyethanol. (BioZone Laboratories Inc., 580 Garcia Avenue, Pittsburg, Calif. USA).

Visits 2, 3 and 4

At each of Visits 2 (Week 4), 3 (Week 8), and 4 (Week 16), information on concomitant illnesses, concomitant medications, and the occurrence of any adverse events were documented. A dermatological examination was conducted by the investigator. The investigator also assigned a global improvement score based on the appearance of the skin of the periorbital area. Subjects completed Self-Assessment Questionnaires. Subject diaries were collected and reviewed, and any unused test products were collected. At Visits 2 and 3, additional four-week supplies of test products were dispensed and new diaries were distributed. At Visit 3 only, photographic images of the facial area, concentrating on the periorbital area, of the subjects were repeated.

At each visit to the study site the investigator evaluated the skin of the periorbital area for:

| tactile laxity/sagging | |
|---|---|
| puffiness | |
| wrinkles | |
| tactile roughness/texture | |
| softness | |
| peeling/flaking | |
| crepiness | |
| dull/matte/sallow appearance | |
| mottled pigmentation | |
| overall aging skin | |
| using the following scale: | |
| 0 = | none |
| 1 = | extremely mild |
| 2 = | very mild |
| 3 = | mild |
| 4 = | mild to moderate |
| 5 = | moderate |
| 6 = | very moderate |
| 7 = | extremely moderate |
| 8 = | moderately severe |
| 9 = | severe |

For qualification, the subject had to have an overall aging skin score of 3-6 in the periorbital area.

Global Improvement Score

At Visits 2, 3 and 4 the investigator evaluated the global improvement of the appearance of the skin of the subject's periorbital area, as compared to baseline (Visit 1) and scored such improvement on an ordinal scale of −1 to 3 as follows:

| | |
|---|---|
| −1 = | worse |
| 0 = | no change |
| 1 = | slightly improved |
| 2 = | improved |
| 3 = | much improved |

Subject's Evaluations (Self-Assessment Questionnaires)

At each visit and at 14 days following Visit 1 subjects self-evaluated their periorbital skin texture for dryness using the following scale:
0=extremely dry
1=very dry
2=dry
3=moderately dry
4=slightly dry
5=slightly moist
6=moderately moist
7=moist
8=very moist
9=extremely moist

At 14 days after the initiation of treatment and at each visit subsequent to Visit 1, subjects self-evaluated improvement in their periorbital skin condition by drawing a vertical line through a 100 mm horizontal visual assessment scale (VAS) with the left end labeled with a “0” and the right end with a “10.” The parameters evaluated are as follows:
periorbital skin sagging [“0”=more sagging and “10”=more lifted];
periorbital skin puffiness [“0”=much more puffy and “10”=much less puffy];
periorbital skin wrinkles [“0” more/deeper wrinkles and “10”=less/smoother wrinkles].

Statistical Methods

Baseline Demographic Analysis

Categorical demographic data, which included race, gender, and age were summarized (refer to Table 1.1).

Efficacy Analysis

Investigator Evaluations

For all skin condition variables and “overall aging,” mean change from baseline scores were greater (i.e., showed greater improvement) for the Soft Gel combination as compared to the placebo combination (refer to Tables 2.1 through 2.4).

Subject Self-Evaluations

For “dryness,” all mean change from baseline scores were greater (i.e., showed greater improvement) for the Soft Gel combination as compared to the placebo combination (refer to Table 2.5 and 2.6).

For “sagging” improvement scores, at Day 14 and Visit 2, mean scores were greater (i.e., showed greater improvement) for the Soft Gel combination as compared to the placebo combination; at Visits 3 and 4, mean scores were greater (i.e., showed greater improvement) for the placebo combination as compared to the Soft Gel combination (refer to Table 2.7).

For “puffiness” improvement scores, at Day 14 and Visits 2 and 3, mean scores were greater (i.e., showed greater improvement) for the Soft Gel combination as compared to the placebo combination; at Visit 4, mean scores were greater (i.e., showed greater improvement) for the placebo combination as compared to the Soft Gel combination (refer to Table 2.7).

For “wrinkling” improvement scores, all mean scores were greater (i.e., showed greater improvement) for the Soft Gel combination as compared to the placebo combination (refer to Table 2.7).

Study Subjects

Disposition of Subjects Completed Versus Discontinued:

There were 23 individuals screened as potential participants in this study. Twenty individuals were qualified and enrolled. Two subjects (Subject Numbers 3 and 12) dropped out, and eighteen subjects subsequently completed the study.

Protocol Deviations

Protocol deviations, which occurred during the course of the study, are listed in Table 1.2. For all subjects, the Day 14 evaluation was completed on Day 15. Five subjects reported eight protocol deviations.

Efficacy Evaluation

Analysis of Baseline Demographic and Clinical Variables

Subjects' demographic data are presented in Table 1.1. No significant differences were observed between the two treatment groups on race, gender, or age. Of the twenty subjects, 100% were female, 85% were Caucasian, with an average age of 54.5 years (range 41-65).

Efficacy Analyses:

TABLE 1.1
Subject Demographics

| | All | Placebo | Soft Gel |
|---|---|---|---|
| | (N = 20) | (n = 10) | (n = 10) |
| Race (n/%) | | | |
| White, Non-Hispanic | 17 | 8 | 9 |
| | (85.0)¹ | (80.0) | (90.0) |
| Hispanic | 3 | 2 | 1 |
| | (15.0) | (20.0) | (10.0) |
| Gender (n/%) | | | |
| Female | 20 | 10 | 10 |
| | (100.0) | (100.0) | (100.0) |
| Age (years) | | | |
| Average | 54.5 | 55.2 | 53.7 |
| SD* | 6.22 | 6.0 | 6.6 |
| Range | 41-65 | 45-65 | 41-65 |

¹Standard Deviation

TABLE 1.2
Protocol Deviations

| Subject # | Description |
|---|---|
| 001-020 | Day 14 - All subject evaluations were |
| | completed on Day 15. |
| 003 | Did not complete a final study visit. |
| | Did not return study product or diaries. |
| 007 | Visit 3 - not completed. Considered a |
| | “missed” visit. |
| 011 | Visit 4 - 4 days late. |
| | Visit 4 - Evaluations completed by |
| | technical staff. PI not available. |
| 012 | Visit 2 - applied prohibited moisturizer |
| | to periorbital area between Visits 1 and 2 |
| | secondary to Adverse Event. |
| 018 | Visit 4 - Dosing diary not returned. |

TABLE 2.1
Investigator Evaluation Scores¹

| | Visit 1 | Visit 2 | Visit 3² | Visit 4 |
|---|---|---|---|---|
| Sagging | | | | |
| Placebo | 4.4 (1.3)³ | 3.6 (1.4) | 3.8 (1.8) | 3.0 (1.9) |
| (N = 8) | | | | |
| Soft Gel | 4.0 (1.2) | 3.7 (1.5) | 3.6 (1.4) | 1.7 (1.5) |
| (N = 10) | | | | |
| Puffiness | | | | |
| Placebo | 3.9 (1.5) | 3.6 (1.6) | 3.4 (1.8) | 2.8 (2.1) |
| (N = 8) | | | | |
| Soft Gel | 3.8 (1.1) | 3.7 (1.3) | 3.4 (1.3) | 1.8 (1.2) |
| (N = 10) | | | | |
| Wrinkles | | | | |
| Placebo | 5.4 (0.5) | 4.3 (0.9) | 4.0 (1.3) | 3.8 (1.2) |
| (N=8) | | | | |
| Soft Gel | 5.4 (0.7) | 4.4 (1.6) | 3.8 (1.5) | 3.0 (1.4) |
| (N = 10) | | | | |
| Roughness | | | | |
| Placebo | 3.5 (0.5) | 3.3 (1.0) | 3.0 (1.1) | 2.6 (1.5) |
| (N = 8) | | | | |
| Soft Gel | 4.0 (0.8) | 3.0 (0.7) | 3.1 (0.8) | 2.1 (0.7) |
| (N = 10) | | | | |
| Softness | | | | |
| Placebo | 3.3 (0.7) | 3.3 (0.7) | 3.0 (1.1) | 2.8 (1.3) |
| (N = 8) | | | | |
| Soft Gel | 3.2 (0.4) | 2.9 (0.6) | 3.0 (0.9) | 1.9 (0.9) |
| (N = 10) | | | | |
| Peeling | | | | |
| Placebo | 2.8 (0.5) | 2.6 (0.9) | 2.1 (0.8) | 1.1 (0.8) |
| (N = 8) | | | | |
| Soft Gel | 2.9 (1.1) | 2.6 (0.7) | 2.0 (0.9) | 0.4 (0.7) |
| (N = 10) | | | | |
| Crepiness | | | | |
| Placebo | 3.4 (0.7) | 3.0 (1.2) | 2.9 (1.6) | 2.4 (1.8) |
| (N = 8) | | | | |
| Soft Gel | 3.2 (0.6) | 3.0 (1.1) | 2.6 (1.1) | 1.0 (1.4) |
| (N = 10) | | | | |
| Sallow | | | | |
| Placebo | 4.1 (1.5) | 3.1 (0.8) | 3.4 (1.4) | 2.8 (1.5) |
| (N = 8) | | | | |
| Soft Gel | 4.3 (0.7) | 3.0 (0.7) | 3.1 (1.1) | 1.4 (1.3) |
| (N = 10) | | | | |
| Mottled | | | | |
| Placebo | 4.1 (1.6) | 3.6 (1.1) | 3.8 (1.3) | 2.9 (1.6) |
| (N = 8) | | | | |
| Soft Gel | 3.7 (1.1) | 3.3 (0.5) | 2.9 (0.9) | 1.6 (1.0) |
| (N = 10) | | | | |
| Overall Aging | | | | |
| Placebo | 4.0 (0.9) | 3.6 (1.2) | 3.4 (1.1) | 3.0 (1.5) |
| (N = 8) | | | | |
| Soft Gel | 4.0 (0.7) | 3.6 (1.2) | 3.0 (1.1) | 2.2 (1.6) |
| (N = 10) | | | | |

¹Scale: 0 = none . . . 3 = mild . . . 6 = very moderate . . . 9 = severe
²For Visit 3 only, Soft Gel N = 9

TABLE 2.2
Investigator Evaluation Scores¹
Change from Baseline²

| | Placebo | Soft Gel | |
|---|---|---|---|
| | (N = 8) | (N = 10) | p-value |
| Sagging | −1.4 | −2.3 | 0.13 |
| | (1.30)³ | (1.16) | |
| Puffiness | −1.1 | −2.0 | 0.20 |
| | (2.03) | (0.47) | |
| Wrinkles | −1.6 | −2.4 | 0.24 |
| | (1.41) | (1.26) | |
| Roughness | −0.9 | −1.9 | 0.06 |
| | (1.36) | (0.74) | |
| Softness | −0.5 | −1.3 | 0.17 |
| | (1.20) | (1.16) | |
| Peeling | −1.6 | −2.5 | 0.07 |
| | (0.74) | (1.08) | |
| Crepiness | −1.0 | −2.2 | 0.04* |
| | (1.31) | (1.03) | |
| Sallow | −1.4 | −2.9 | 0.03* |
| | (1.30) | (1.37) | |
| Mottled | −1.3 | −2.1 | 0.29 |
| | (1.49) | (1.73) | |
| Overall Aging | −1.0 | −1.8 | |
| | (1.31) | (1.32) | 0.22 |

¹Scale: 0 = none . . . 3 = mild . . . 6 = very moderate . . . 9 = severe
²Change from Visit 1 to Visit 4
³Standard Deviation
*Statistically significant, p < 0.05

TABLE 2.3
Investigator Global Improvement Scores¹

| | | |
|---|---|---|
| Visit 2 | Visit 3² | Visit 4 |

Global Improvement

| | | | |
|---|---|---|---|
| Placebo | 0.6 (0.5)³ | 1.0 (1.2) | 1.0 (1.2) |
| (N = 8) | | | |
| Soft Gel | 0.7 (0.7) | 1.0 (0.9) | 2.1 (1.2) |
| (N = 10) | | | |

¹Scale: −1 = worse . . . 1 = slightly improved . . . 3 = much improved
²For Visit 3 only, Soft Gel N = 9
³Standard Deviation

TABLE 2.4
Investigator Global Improvement Scores¹
Change from Visit 2²

| | Placebo | Soft Gel | |
|---|---|---|---|
| | (N = 8) | (N = 10) | p-value |
| Global Improvement | 0.4 | 1.4 | 0.02* |
| | (0.9)³ | (0.7) | |

¹Scale: 0 = none . . . 3 = mild . . . 6 = very moderate . . . 9 = severe
²Change from Visit 2 to Visit 4
³Standard Deviation
*Statistically significant, p < 0.05

TABLE 2.5
Subject Dryness Scores¹

| | | | | |
|---|---|---|---|---|
| Visit 1 | Day 14 | Visit 2 | Visit 3² | Visit 4 |

Dryness

| | | | | | |
|---|---|---|---|---|---|
| Placebo | 3.1 | 4.3 | 4.4 | 4.9 | 4.8 |
| (N = 8) | (1.6)³ | (1.4) | (1.3) | (1.6) | (1.8) |
| Soft Gel | 3.2 | 5.2 | 5.4 | 5.4 | 5.3 |
| (N = 10) | (0.8) | (1.5) | (1.3) | (1.4) | (1.7) |
| | p = 0.22⁴ | p = 0.22 | p = 0.22 | p = 0.22 | p = 0.22 |

¹Scale: 0 = extremely dry . . . 3 = moderately dry . . . 6 = moderately moist . . . 9 = extremely moist
²For Visit 3 only, Soft Gel N = 9
³SD = Standard Deviation
⁴p = significance of the difference between groups

TABLE 2.6
Subject Dryness Scores¹
Change from Baseline²

| | Placebo | Soft Gel | |
|---|---|---|---|
| | (N = 8) | (N = 10) | p-value |
| Dryness | 1.6 | 2.1 | |
| | (1.6)³ | (2.2) | 0.62 |

¹Scale: 0 = extremely dry . . . 9 = extremely moist
²Change from Visit 1 to Visit 4
³SD = Standard Deviation

TABLE 2.7
Subject Improvement Evaluation Scores¹

| | Day 14 | Visit 2 | Visit 3² | Visit 4 |
|---|---|---|---|---|
| Sagging | | | | |
| Placebo | 1.4 | 2.0 | 4.0 | 4.5 |
| (N = 8) | (1.2)³ | (1.6) | (2.4) | (2.7) |
| Soft Gel | 2.8 | 3.2 | 3.6 | 4.0 |
| (N = 10) | (2.8) | (3.0) | (3.0) | (3.0) |
| | p = 0.22⁴ | p = 0.31 | p = 0.78 | p = 0.73 |
| Puffiness | | | | |
| Placebo | | | | |
| (N = 8) | 1.4 | 1.7 | 3.7 | 4.4 |
| | (1.7) | (1.9) | (2.7) | (2.0) |
| SoftGel | | | | |
| (N = 10) | 2.7 | 3.9 | 3.9 | 4.1 |
| | (2.6) | (3.3) | (3.2) | (3.3) |
| | p = 0.25 | p = 0.11 | p = 0.87 | p = 0.81 |
| Wrinkles | | | | |
| Placebo | | | | |
| (N = 8) | 1.2 | 1.8 | 3.8 | 4.1 |
| | (1.1) | (1.4) | (2.5) | (2.5) |
| SoftGel | | | | |
| (N = 10) | 2.9 | 3.7 | 4.3 | 4.5 |
| | (2.7) | (3.0) | (3.4) | (3.1) |
| | p = 0.14 | p = 0.12 | p = 0.73 | p = 0.81 |

¹Scale: 0 = [more sagging, more puffy, more wrinkles] . . . 10 = [more lifted, less puffy, smoother]
²For Visit 3 only, Soft Gel N = 9
³SD = Standard Deviation
⁴p = significance of the difference between groups

TABLE 2.8
Subject Improvement Evaluation Change Scores¹

| | Day 14 to | Visit 2 to | Visit 3² to |
|---|---|---|---|
| | Visit 2 | Visit 3² | Visit 4 |
| Sagging | | | |
| Placebo | 0.6 | 2.0 | 0.5 |
| (N = 8) | (0.8)³ | (1.7) | (1.4) |
| Soft Gel | 0.4 | 0.3 | 0.9 |
| (N = 10) | (0.8) | (2.1) | (1.6) |
| | p = 0.76 | p = 0.09 | p = 0.67 |
| Puffiness | | | |
| Placebo | 0.3⁴ | 2.0 | 0.7 |
| (N = 8) | (0.7) | (2.7) | (1.8) |
| SoftGel | 1.2 | −0.1 | 0.6 |
| (N = 10) | (1.3) | (2.0) | (1.4) |
| | p = 0.08 | p = 0.08 | p = 0.89 |
| Wrinkles | | | |
| Placebo | 0.6 | 2.0 | 0.3 |
| (N = 8) | (0.4) | (0.4) | (1.4) |
| SoftGel | 0.8 | 0.4 | 0.7 |
| (N = 10) | (0.9) | (2.7) | (2.1) |
| | p = 0.42 | p = 0.20 | p = 0.72 |

¹Scale: 0 = [more sagging, more puffy, more wrinkles] . . . 10 = [more lifted, less puffy, smoother]
²For Visit 3 only, Soft Gel N = 9
³SD = Standard Deviation
⁴p = significance of the difference between groups

Investigator Evaluations

Tactile Laxity/Sagging

The mean “tactile laxity/sagging” scores at baseline for the Placebo and Soft Gel groups were 4.4 and 4.0, respectively (refer to Table 2.1). The mean change from baseline scores for the Placebo and Soft Gel groups were −1.4 and −2.3, respectively; these differences were not found to be statistically significant, p=0.13 (refer to Table 2.2).

Puffiness

The mean “puffiness” scores at baseline for the Placebo and Soft Gel groups were 3.9 and 3.8, respectively (refer to Table 2.1). The mean change from baseline scores for the Placebo and Soft Gel groups were −1.1 and −2.0, respectively; these differences were not found to be statistically significant, p=0.20 (refer to Table 2.2).

Wrinkles

The mean “wrinkles” scores at baseline for the Placebo and Soft Gel groups were 5.4 and 5.4, respectively (refer to Table 2.1). The mean change from baseline scores for the Placebo and Soft Gel groups were −1.6 and −2.4, respectively; these differences were not found to be statistically significant, p=0.24 (refer to Table 2.2).

Tactile Roughness/Texture

The mean “tactile roughness/texture” scores at baseline for the Placebo and Soft Gel groups were 3.5 and 4.0, respectively (refer to Table 2.1). The mean change from baseline scores for the Placebo and Soft Gel groups were −0.9 and −1.9, respectively; these differences approached statistical significance, p=0.06 (refer to Table 2.2).

Softness

The mean “softness” scores at baseline for the Placebo and Soft Gel groups were 3.3 and 3.2, respectively (refer to Table 2.1). The mean change from baseline scores for the Placebo and Soft Gel groups were −0.5 and −1.3, respectively; these differences were not found to be statistically significant, p=0.17 (refer to Table 2.2).

Peeling/Flaking

The mean “peeling/flaking” scores at baseline for the Placebo and Soft Gel groups were 2.8 and 2.9, respectively (refer to Table 2.1). The mean change from baseline scores for the Placebo and Soft Gel groups were −1.6 and −2.5, respectively; these differences approached statistical significance, p=0.07 (refer to Table 2.2).

Crepiness

The mean “crepiness” scores at baseline for the Placebo and Soft Gel groups were 3.4 and 3.2, respectively (refer to Table 2.1). The mean change from baseline scores for the Placebo and Soft Gel groups were −1.0 and −2.2, respectively; these differences were found to be statistically significant, p=0.04 (refer to Table 2.2).

Dull/Matte/Sallow Appearance

The mean “dull/matte/sallow appearance” scores at baseline for the Placebo and Soft Gel groups were 4.1 and 4.3, respectively (refer to Table 2.1). The mean change from baseline scores for the Placebo and Soft Gel groups were −1.4 and −2.9, respectively; these differences were found to be statistically significant, p=0.03 (refer to Table 2.2).

Mottled Pigmentation

The mean “mottled pigmentation” scores at baseline for the Placebo and Soft Gel groups were 4.1 and 3.7, respectively (refer to Table 2.1). The mean change from baseline scores for the Placebo and Soft Gel groups were −1.3 and −2.1, respectively; these differences were not found to be statistically significant, p=0.29 (refer to Table 2.2).

Overall Aging Skin

The mean “overall aging skin” scores at baseline for the Placebo and Soft Gel groups were 4.0 and 4.0, respectively (refer to Table 2.1). The mean change from baseline scores for the Placebo and Soft Gel groups were −1.0 and −1.8, respectively; these differences were not found to be statistically significant, p=0.22 (refer to Table 2.2).

Global Improvement

The mean “global improvement” scores at Visit 2 for the Placebo and Soft Gel groups were 0.6 and 0.7, respectively (refer to Table 2.3). The mean change from Visit 2 scores for the Placebo and Soft Gel groups were 0.4 and 1.4, respectively; these differences were found to be statistically significant, p=0.02 (refer to Table 2.4).

Subject Self-Assessments

Dryness of the Periorbital Skin

The mean “dryness” scores at baseline for the Placebo and Soft Gel groups were 3.1 and 3.2, respectively (refer to Table 2.5). The mean change from baseline scores for the Placebo and Soft Gel groups were 1.6 and 2.1, respectively; these differences were not found to be statistically significant, p=0.62 (refer to Table 2.6).

Improvement in Periorbital Skin Condition Sagging

The mean “sagging” improvement scores for the Placebo and Soft Gel groups, respectively, were 1.4 and 2.8, at Day 14 (p=0.22); 2.0 and 3.2 at Visit 2 (p=0.31); 4.0 and 3.6 at Visit 3 (p=0.78); and 4.5 and 4.0 at Visit 4 (p=0.73) [refer to Table 2.7]. The mean “sagging” improvement change scores for the Placebo and Soft Gel groups, respectively, were 0.6 and 0.4, for Day 14 to Visit 2 (p=0.76); 2.0 and 0.3 for Visit 2 to Visit 3 (p=0.09); 0.5 and 0.9 for Visit 3 to Visit 4 (p=0.67) [refer to Table 2.8].

Puffiness

The mean “puffiness” improvement scores for the Placebo and Soft Gel groups, respectively, were 1.4 and 2.7, at Day 14 (p=0.25); 1.7 and 3.9 at Visit 2 (p=0.11); 3.7 and 3.9 at Visit 3 (p=0.87); and 4.4 and 4.1 at Visit 4 (p=0.81) [refer to Table 2.7]. The mean “puffiness” improvement change scores for the Placebo and Soft Gel groups, respectively, were 0.3 and 1.2, for Day 14 to Visit 2 (p=0.08); 2.0 and −0.1 for Visit 2 to Visit 3 (p=0.08); 0.7 and 0.6 for Visit 3 to Visit 4 (p=0.89) [refer to Table 2.8].

Wrinkles

The mean “wrinkles” improvement scores for the Placebo and Soft Gel groups, respectively, were 1.2 and 2.9, at Day 14 (p=0.14); 1.8 and 3.7 at Visit 2 (p=0.12); 3.8 and 4.3 at Visit 3 (p=0.73); and 4.1 and 4.5 at Visit 4 (p=0.81) [refer to Table 2.7]. The mean “wrinkles” improvement change scores for the Placebo and Soft Gel groups, respectively, were 0.6 and 0.8, for Day 14 to Visit 2 (p=0.42); 2.0 and 0.4 for Visit 2 to Visit 3 (p=0.20); 0.3 and 0.7 for Visit 3 to Visit 4 (p=0.72) [refer to Table 2.8].

Efficacy Conclusions:

This trial demonstrated that the Soft Gel oral and topical products combination was safe and effective in improving skin appearance and condition (crow's feet/skin elasticity) in the periorbital area.

Investigator Evaluations

In all skin condition categories of the Investigator evaluation of the skin of the periorbital area (tactile laxity/sagging, puffiness, wrinkles, tactile roughness/texture, softness, peeling/flaking, crepiness, dull/matte/sallow appearance, mottled pigmentation, overall aging skin) the Soft Gel products combination was rated as demonstrating greater improvement than the placebo products combination. In three categories (crepiness, dull/matte/sallow appearance, and global improvement) the differences in improvement ratings between the treatment groups was statistically significant in favor of the Soft Gel products combination. In two other categories (tactile roughness/texture, peeling/flaking) differences, favoring the Soft Gel combination, approached significance. The global improvement scores were greater for the Soft Gel combination; the difference in the change from baseline (Visit 2) scores for global improvement were statistically significant in favor of the Soft Gel products combination.

Subject Self-Assessments

In the category of “dryness,” at each time point subject self-assessments rated the Soft Gel combination as demonstrating greater improvement than the placebo combination, however the differences were not statistically significant.

In the categories of “sagging,” “puffiness,” and “wrinkling,” subject self-ratings at Day 14 showed that the Soft Gel combination resulted in greater improvement as compared to the placebo, however, these differences were found to be statistically significant. Change scores for “sagging” showed that only at Visit 3 was the Soft Gel change score greater than the placebo change score. Change scores for “puffiness” showed that only at Visit 2 was the Soft Gel change score greater than the placebo change score. Change scores for “wrinkles” showed that at Visits 2 and 4 the change scores for Soft Gel were greater than the placebo change scores. The differences were not found to be statistically significant.

The evaluations demonstrated that the Soft Gel oral and topical products combination was effective in providing greater improvement for the appearance and condition of aging skin in the periorbital area when compared to a placebo products combination. The Soft Gel combination resulted in greater improvement in all skin appearance and condition categories and in global improvement. In three categories, “crepiness,” “dull/matte/sallow appearance,” and “global improvement” the between group differences were found to be statistically significant in favor of the Soft Gel combination. In other categories, “tactile roughness/texture” and “peeling/flaking,” there was borderline significance observed in favor of the Soft Gel products combination. Subject self-assessments of “dryness” showed Soft Gel achieving greater improvement scores. For subject self-assessments of “sagging,” “puffiness,” and “wrinkling,” there was more variation in individual values with the Soft Gel combination achieving greater improvement scores at nine out of twelve time points.

The results of this clinical trial, in which 20 subjects with mild to very moderate aging skin in the periorbital area participated, demonstrated that the Soft Gel oral and topical products combination demonstrated greater improvement in skin appearance and condition, and greater global improvement, as observed by the investigator, when compared to a placebo products combination. Subject self-assessments showed that the Soft Gel products combination resulted in greater improvement in “dryness,” and at nine out of twelve time points for “sagging,” “puffiness,” and “wrinkling. No serious adverse events were reported during this study. With a larger sample size, the trends observed in this trial towards significance would be expected to continue and to favor the Soft Gel products combination. Overall, the Soft Gel products combination was found to be safe and effective when used to treat the appearance and condition of aging skin in the periorbital area.

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

1. An ingestible composition comprising:
a processed ceramide or an analog thereof; and
an acceptable carrier.

2. The composition of claim 1, with the provisio the carrier is not a silicone polymer.

3. The composition of claim 1, wherein said ceramide is a phyto-derived ceramide.

4. The composition of claim 1, further comprising hyaluronic acid.

5. The composition of claim 1, further comprising biotin.

6. The composition of claim 1, further comprising hyaluronic acid and biotin.

7. The composition of claim 1, wherein said carrier is wheat germ oil or yellow beeswax.

8. The composition of claim 1, wherein said carrier is wheat germ oil and yellow beeswax.

9. A method for oral delivery of an effective amount of a ceramide to an individual in need thereof, comprising the steps of:
providing a composition comprising a processed ceramide or an analog thereof and an acceptable carrier suitable for oral administration, such that an effective amount of ceramide is provided to an individual.

10. A packaged nutraceutical formulation for oral administration of a ceramide comprising:
a composition comprising a processed ceramide or an analog thereof and a carrier; and
instructions for oral administration thereof.

11. A method to treat dry skin, comprising the step of ingesting a composition comprising a processed ceramide or an analog thereof and an acceptable carrier.

12. A method to treat wrinkles, comprising the step of ingesting a composition comprising a processed ceramide and an acceptable carrier.

13. An ingestible soft gelatin capsule comprising:
a processed ceramide or an analog thereof; and
an acceptable carrier.

14. The soft gelatin capsule of claim 13, with the provisio the carrier is not a silicone polymer.

15. The soft gelatin capsule of claim 13, wherein said processed ceramide is a phyto-derived ceramide.

16. The soft gelatin capsule of claim 13, further comprising hyaluronic acid.

17. The soft gelatin capsule of claim 13, further comprising biotin.

18. The soft gelatin capsule of claim 13, further comprising hyaluronic acid and biotin.

19. The soft gelatin of claim 13, wherein said carrier is wheat germ oil or yellow beeswax.

20. The soft gelatin capsule of claim 13, wherein said carrier is wheat germ oil and yellow beeswax.

21. A method for the delivery of an effective amount of a ceramide to an individual in need thereof, comprising the steps of
providing a processed ceramide or an analog thereof and an acceptable carrier in a soft gelatin capsule suitable for ingestion, such that an effective amount of ceramide is provided to an individual.

22. A packaged nutraceutical formulation for oral administration of a ceramide comprising:
a soft gelatin capsule comprising a processed ceramide or an analog thereof and a carrier; and
instructions for oral administration thereof.

23. A method to treat dry skin, comprising the step of ingesting a soft gel capsule comprising a processed ceramide or an analog thereof and an acceptable carrier.

24. A method to treat wrinkles, comprising the step of ingesting a soft gelatin capsule comprising a processed ceramide and an acceptable carrier.
